(19) United States
(12) Patent Application Publication
(10) Pub. No.: US 2023/0029744 A1
(43) Pub. Date: Feb. 2, 2023

(54) INTRACRANIAL DIAGNOSTICS USING OPTICAL IMAGING OF COHERENT LIGHT INTERFERENCE

(71) Applicant: Open Water Internet Inc., San Francisco, CA (US)

(72) Inventors: Soren Konecky, Alameda, CA (US); Brad Hartl, San Mateo, CA (US); Kedar Grama, Sausalito, CA (US); Achal Singh Achrol, Highland, CA (US)

(21) Appl. No.: 17/387,279

(22) Filed: Jul. 28, 2021

(51) Int. Cl. A61B 5/00; G06T 7/00; G06T 7/20; G06K 9/20; G08B 7/06; A61B 5/026

(52) U.S. Cl. CPC A61B 5/0042; G06T 7/0016; G06T 7/20; G06K 9/209; G08B 7/06; A61B 5/0261; G06T 2207/30104; G06T 2207/10016; A61B 2576/026

(57) ABSTRACT

Coherent light (e.g., laser light) is emitted into a cranium through an optical fiber. A tissue sample (e.g., red blood cells, blood vessels, brain tissue) within the cranium diffuses the coherent light. Different tissue sample motion quantities generate different coherent light interference patterns. An image of a coherent light interference pattern is captured with an image sensor coupled to an optical element. The speckle contrast of the image quantifies coherent light interference pattern. A waveform of sequentially captured speckle contrast values over time has characteristics that reflect intracranial blood flow health. If waveform characteristics indicate poor or questionable intracranial blood flow heath, a notification message is displayed, played, or otherwise transmitted.

### BACKGROUND INFORMATION

Imaging devices are used in contexts such as healthcare, navigation, and security, among others. Imaging systems often measure radio waves or light waves to facilitate imaging. Imaging that measures light scattered by an object is especially challenging and advances to the devices, systems, and methods to improve optical imaging are sought to increase speed, increase resolution, reduce size and/or reduce cost.

In the context of healthcare, intrusive procedures are regularly used to evaluate the intracranial health (e.g., the intracranial pressure) of a patient. For example, a common practice is for medical professionals bore a hole through a patient's cranium to facilitate the continued acquisition of data through the use of intracranial transducers. While accurate, the use of transducers in this manner incurs cost to the patient, introduces the patient to potential infection, and requires additional care or caretaker support for the patient. Currently, alternatives to transducer-based evaluation of intracranial health include MRI or CT scanning techniques, which are financially impractical for continued, real-time data acquisition.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 illustrates an imaging system configured to determine intracranial motion characteristics of a tissue sample, in accordance with aspects of the disclosure.

FIG. 2 illustrates an example light detector having an image sensor configured to image an interference pattern generated by a reference beam interfering with a measurement beam, in accordance with aspects of the disclosure.

FIG. 3 illustrates an example configuration of a light detector including a beam splitter, in accordance with aspects of the disclosure.

FIGS. 4A-4F illustrate example graphs of intracranial motion characteristics and blood characteristics that may be incorporated into data models and rule sets, in accordance with aspects of the disclosure.

FIG. 4G illustrates a prior art graph of a normal example of an intracranial pressure waveform.

FIGS. 5A-5B illustrate an image pixel array coupled to processing logic configured to generate composite images and notifications of blood flow performance, in accordance with aspects of the disclosure.

FIG. 6 illustrates an imaging system including a network of light detectors configured to determine intracranial motion characteristics, in accordance with aspects of the disclosure.

FIG. 7 illustrates an imaging system applied to a human head and configured to determine intracranial motion characteristics, in accordance with aspects of the disclosure.

FIG. 8 illustrates a display of a composite image of blood flow characteristics, in accordance with aspects of the disclosure.

FIG. 9 illustrates a flow diagram of a process for determining intracranial motion characteristics from coherent light interference patterns, in accordance with aspects of the disclosure.

FIG. 10 illustrates a flow diagram of a process for determining intracranial motion characteristics from coherent light interference patterns, in accordance with aspects of the disclosure.

### DETAILED DESCRIPTION

Embodiments of optical imaging with light coherence are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.4 μm.

This disclosure will generally describe imaging a diffuse medium in the context of human tissue in the medical context. However, the content of this disclosure may be applied to medical imaging, navigation, security, scientific research, or other contexts that image diffuse mediums or objects.

Human tissue is translucent to infrared light, although different parts of the human body (e.g., skin, blood, bone) exhibit different absorption and scattering coefficients. Researchers have attempted to use the properties of infrared light for medical imaging purposes, but size and cost constraints have been prohibitive for wide-scale adoption. Illuminating tissue and other diffuse media with near-infrared light for imaging purposes is sometimes referred to as Diffuse Optical Tomography. In one optical technique, Laser Speckle Imaging can be used to detect light primarily reflected near the surface of a sample, severely lacking depth of measurement. In another Diffuse Optical Tomography technique, Diffuse Correlation Spectroscopy uses an avalanche photodiode to measure coherence by looking at a single speckle over time. However, a single speckle provides limited information on the movement of fluid or overall motion of a sample.

In contrast to Laser Speckle Imaging and Diffuse Correlation Spectroscopy, some embodiments of this disclosure may include an imaging system that may be configured to emit laser light through a first optical fiber into a cranium, detect diffused light from the tissue sample through a second optical fiber, capture images of the diffused light, determine coherence values for the images, generate a waveform of the coherence values, and determine intracranial motion characteristics at least partially based on various characteristics of the waveform of coherence values. The imaging system may emit laser light using one or more coherent light sources having one or more optical fibers coupled to one or more coherent light sources. The imaging system may detect diffused light using one or more light detectors having one or more optical fibers coupled to one or more image sensors. Intracranial motion characteristics may include blood cell motion, blood vessel motion, brain tissue motion, and/or cranial vibrations. Characteristics of the waveform of coherence values may include the number of valleys in a cycle of the waveform, relative values of valleys in a cycle of the waveform, and the relative shape of a waveform captured from a first location on the cranium compared to the shape of a waveform captured from a second location on the cranium. The imaging system may determine intracranial motion characteristics from the waveform(s) by using processing logic coupled to the light sources and light detectors.

The processing logic may use coherent light interference patterns represented in the image to determine intracranial motion characteristics. Coherent light includes, but is not limited to, light waves or photons having the same frequency, phase, and polarization. Coherent light interference in an image may be manifest or captured as speckles, which include bright and dark spots of one or more pixels in an image. Dark pixels are pixels that have a lower pixel value than surrounding pixels and/or than the average pixel value of an image. Bright pixels are pixels that have a higher pixel value than surrounding pixels and/or than the average pixel value of an image. Speckles, and therefore coherent light interference, in an image may be quantified as a coherence value of an image.

A coherence value can represent one of a number of different metrics. A coherence value can be a variance of pixels of an image, a standard deviation of pixels of an image, and/or a speckle contrast of pixels of an image. Speckle contrast is a measure of the coherence of light hitting a light detector, e.g., an image sensor. Speckle contrast may be determined by dividing the standard deviation of the pixel values of an image by the mean of the pixel values of an image (i.e., std/mean). The coherence values of sequentially captured images are combined to form a waveform representing intracranial motion. Higher coherence values represent less motion within the cranium. Lower coherence values represent more motion within the cranium.

The processing logic applies rules or data models to the waveform of coherence values to identify potential reductions in intracranial blood flow (e.g., as might be manifest by an ischemic or hemorrhagic stroke). The rules or data models may define waveform characteristics that are indicative of potential reductions in intracranial blood flow. Examples of rules include: within a cycle of the waveform, is a second valley value less than or equal to a first valley value; is a cycle limited to a single valley and single peak; and the number of valleys in a cycle of a waveform captured from one location on the cranium differs from the number of valleys in a cycle of a waveform captured from another location on the cranium.

The processing logic may be configured to apply rules or data models to an inverted waveform of coherence values to identify potential reductions in intracranial blood flow (e.g., as might be manifest by an ischemic or hemorrhagic stroke). An inverted waveform of coherence values resembles an intracranial pressure (“ICP”) waveform. A first peak, a second peak, and a third peak of a cycle of an inverted waveform of coherence values may be analyzed as though they were a percussion wave, a tidal wave, and a dicrotic wave of an ICP waveform, respectively. Accordingly, the rules or data models may be defined around inverted waveform characteristics that are indicative of potential reductions in intracranial blood flow. Examples of rules include: within a cycle of the inverted waveform, is a second peak value greater than or equal to a first peak value; is a cycle limited to a single valley and single peak (i.e., the second and third peaks are indistinguishable from the first peak); and the number of peaks in a cycle of an inverted waveform captured from one location on the cranium differs from the number of peaks in a cycle of an inverted waveform captured from another location on the cranium.

The processing logic may include a notification system that generates a notification in response to identifying a potential reduction of intracranial blood flow. The notification system may include output notifications to a display, a speaker, a haptic motor, and/or a computing device to notify a person that intracranial blood flow is reduced, unknown, and/or typical, for example.

Embodiments of the imaging system of this disclosure may include various configurations. The imaging system may include multiple light sources, multiple optical fibers, multiple lasers, continuous wave lasers, pulsed lasers, and/or continuous wave laser light that is modulated or chopped. The imaging system may include direct capture of image data from an optical fiber, or may include optically combined captured light with a reference light source. The imaging system may use speckle contrast, interference with a reference beam, and/or optical attenuation to determine blood characteristics within a tissue sample. Various types of data models may be employed to decipher meaning from an image (e.g., speckle contrast).

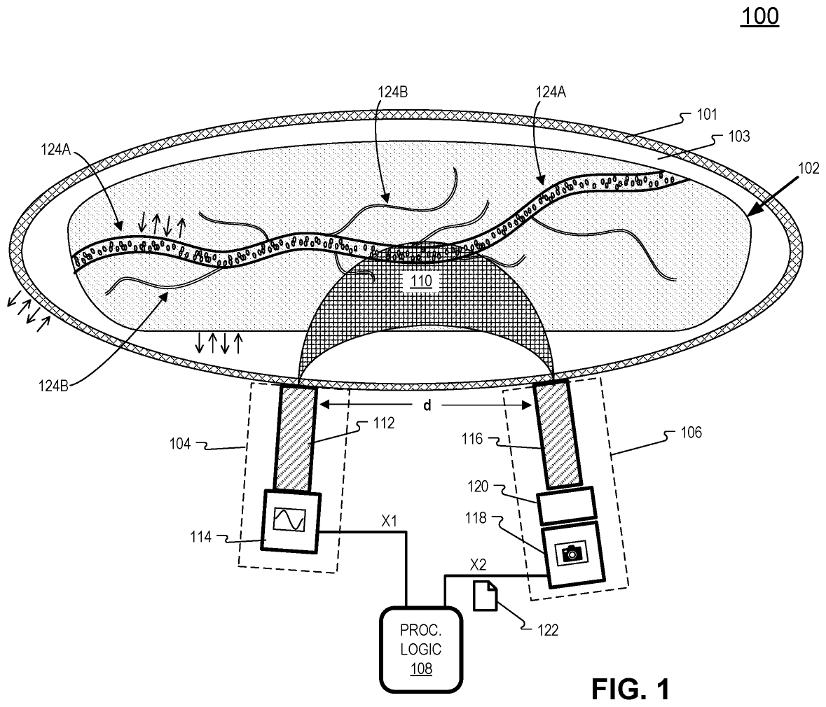

These embodiments and others will be described in more detail with reference to FIGS. 1-10.

FIG. 1 illustrates an imaging system 100 that is configured to determine intracranial motion characteristics in a cranium 101 and for a tissue sample 102, at least partially based on the coherence of diffuse light captured in a sequence of images, in accordance with aspects of the disclosure. The coherence of diffuse light in a captured image is manifest in the image as coherent light interference patterns, or speckles. The coherent light interference (or interference patterns) may be converted to a coherence value and may be quantified by determining the variance, standard deviation, and/or speckle contrast of an image of the diffuse light exiting cranium 101. Sequentially captured coherence values may be combined into a waveform, which may be correlated with intracranial motion characteristics. Examples of intracranial motion characteristics include, but are not limited to, blood cell motion (e.g., flow rates), blood vessel motion (e.g., displacement, dilation, and constriction), brain tissue motion (e.g., displacement), and cranial vibration. The waveform of coherence values may therefore be used to identify blood vessel occlusions, which may be indicative of health issues (e.g., an ischemic stroke). Imaging system 100 may include a light source 104, a light detector 106, and processing logic 108 that are configured to measure intracranial motion characteristics in cranium 101 and tissue sample 102. Various features of imaging system 100 are described in further detail below.

Light source 104 is configured to emit light into cranium 101 as measurement beam 110. Light source 104 includes a source optical fiber 112 and a light generator 114 coupled to source optical fiber 112. Source optical fiber 112 is positioned against cranium 101 to provide a path for photons to travel between light generator 114 and tissue sample 102. Light generator 114 is configured to generate coherent light of a narrow band of frequencies. Light generator 114 may be a laser source configured to emit near-infrared laser light. In one embodiment, the near-infrared laser light has a wavelength between 700 nm and 1000 nm. In one embodiment, the laser light has a wavelength of 600 nm to 900 nm. The laser light may provide a narrow band of coherent light at approximately 850 nm, for example. The laser may be a continuous wave (CW) laser. The output of the laser may be pulsed, chopped, or modulated to provide pulses of coherent light. The pulses may have a duration of 10 μs, 20 μs, or some other duration from 10 μs to 1000 μs, according to various implementations.

Light detector 106 is configured to detect coherent light from measurement beam 110, which is formed from the coherent light diffused into tissue sample 102 by light source 104. Light detector 106 may include a detector optical fiber 116 coupled to an image sensor 118. Detector optical fiber 116 may be a multi-mode optical fiber having a core diameter of 50 μm, 60 μm, or some diameter greater than approximately 10 μm. Source optical fiber 112 may be a single-mode optical fiber having a diameter of 9 μm or less. In one embodiment, detector optical fiber 116 is implemented as another optical element, such as a window or one or more lenses position within light detector 106.

Detector optical fiber 116 captures diffused light (i.e., an exit signal) from cranium 101 and transports the diffused light from the measurement beam 110 to image sensor 118. Image sensor 118 may be a complementary metal oxide semiconductor (“CMOS”) image sensor or a charge-coupled device (“CCD”) image sensor. Image sensor 118 includes an array of pixels that are each responsive to photons received from measurement beam 110 through detector optical fiber 116. Pixels in image sensor 118 respond to interference of coherent light with dark pixels values and bright pixels values that manifest in an image as speckles. A coherence value for the image quantifies the speckles in the image, which is used to determine intracranial motion characteristics within cranium 101. In one embodiment, image sensor 118 has image sensor pixels having a pixel pitch of one micron or less. The pixel resolution of image sensor 118 may vary depending on the application. In one embodiment, image sensor 118 is 1920 pixels by 1080 pixels. In one embodiment, image sensor 118 is a 40 megapixel or greater image sensor.

In an embodiment, a light converter 120 is positioned between detector optical fiber 116 and image sensor 118 to facilitate transmission of light between light detection optical fiber 116 and image sensor 118. Light converter 120 may be implemented as one or more of a lens, a filter, and/or an optical switch, in an embodiment. Light converter 120 may include a bandpass filter. Light converter 120 may be a high-pass filter that filters out ambient light wavelengths.

Processing logic 108 is coupled to light source 104 and light detector 106 to support operation of the imaging system 100, according to an embodiment. Processing logic 108 uses channel X1 to send control signals to light source 104 to operate light source 104. Examples of operating light source 104 include turning light generator 114 on and off and include chopping and/or pulsing the output of light generator 114.

Processing logic 108 uses channel X2 to send control signals to image sensor 118, in an embodiment. Processing logic 108 may configure the exposure time of the image sensor 118. Examples of the exposure time include 10 μs, 20 μs, 30 μs, or various increments in the range of 10 μs to 1000 μs. The strength of the coherence value (e.g., speckle contrast) signal may decrease with increasing exposure times, e.g., greater than 100 μs. Therefore, in some implementations, exposure time for image sensor 118 is configured to be less than 100 μs.

Processing logic 108 uses channel X2 to receive image data 122 from image sensor 118, in an embodiment. Image data 122 may include an array of pixel values representing exposure of the pixel array of image sensor 118 to photons from measurement beam 110. Measurement beam 110 is the portion of light emitted by light source 104 that exits into light detector 106. The portion of measurement beam 110 that exits cranium 101 into light detector 106 may be referred to as an exit signal. When light source 104 is a laser, measurement beam 110 includes laser light emitted by light source 104 into cranium 101 that at least partially propagates to light detector 106. The diffused light of measurement beam 110 may take a more round-about optical path than is illustrated in FIG. 1. Measurement beam 110 has a depth into cranium 101 that at least partially depends on the distance d between light source 104 and light detector 106. That is, a larger distance d enables/targets deeper measurements and a smaller distance d enables/targets shallower measurements. Processing logic 108 may use channel X2 to receive image data 122 for speckle contrast analysis or other coherence value determination.

Intracranial motion characteristics may include parameters that quantify motion of blood cells, blood vessels, brain tissue, cranial fluid, and/or cranium 101. Motion of blood cells may be represented as blood flow rates, blood volume, and/or blood oxygenation. Motion of blood vessels 124 is depicted in FIG. 1 with arrows pointed in opposite directions. Motion of blood vessels 124 may be caused by blood vessel displacement, dilation, or constriction during, for example, systolic uptake, systolic decline, diastolic runoff, aortic valve closure, or the like. Motion of (brain) tissue sample 102 is depicted in FIG. 1 with arrows pointed in opposite directions. Brain tissue may displace, pulsate, or otherwise move in response to blood flow cycles. Cranial fluid 103 may transfer motion energy to cranium 101. Motion of cranium 101 is depicted in FIG. 1 with arrows pointed in opposite directions and may be manifest as vibrations detected by imaging system 100.

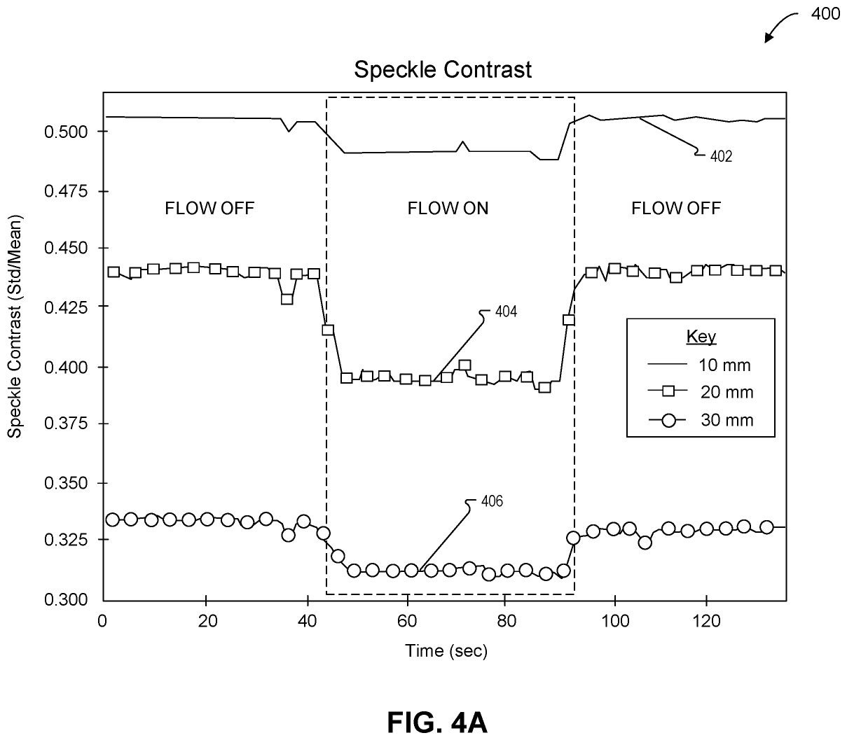

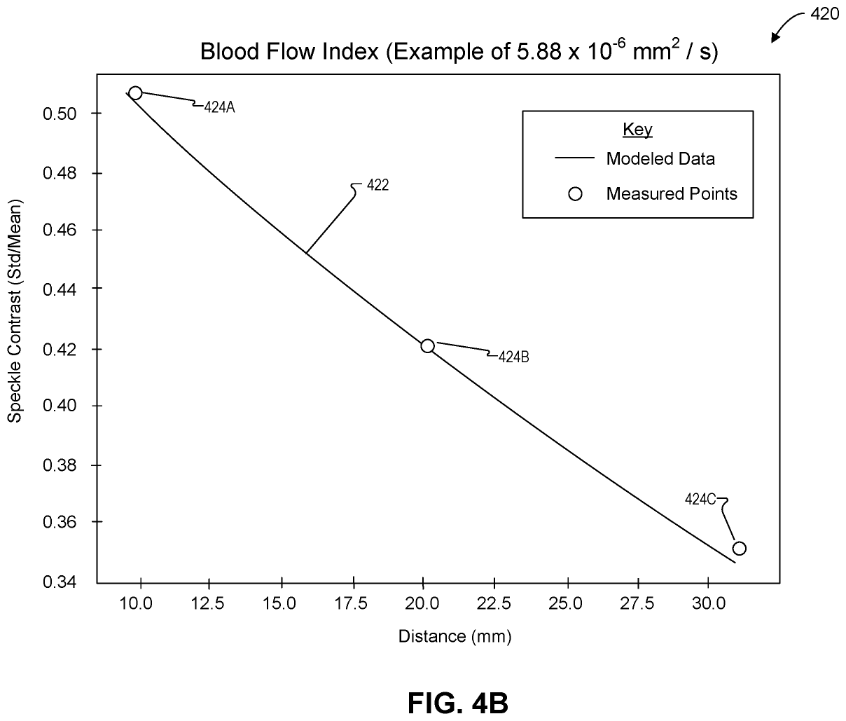

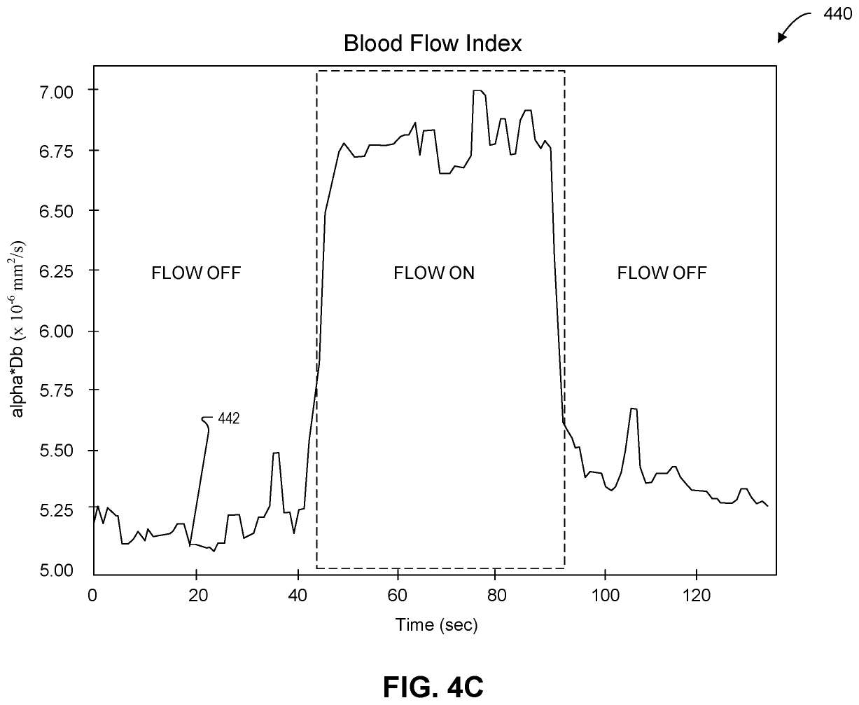

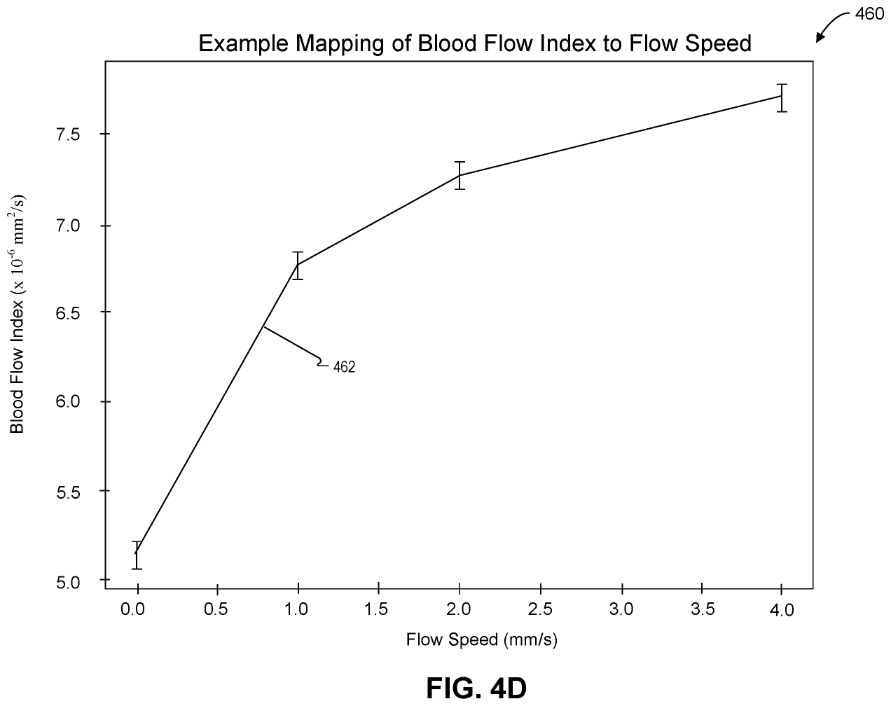

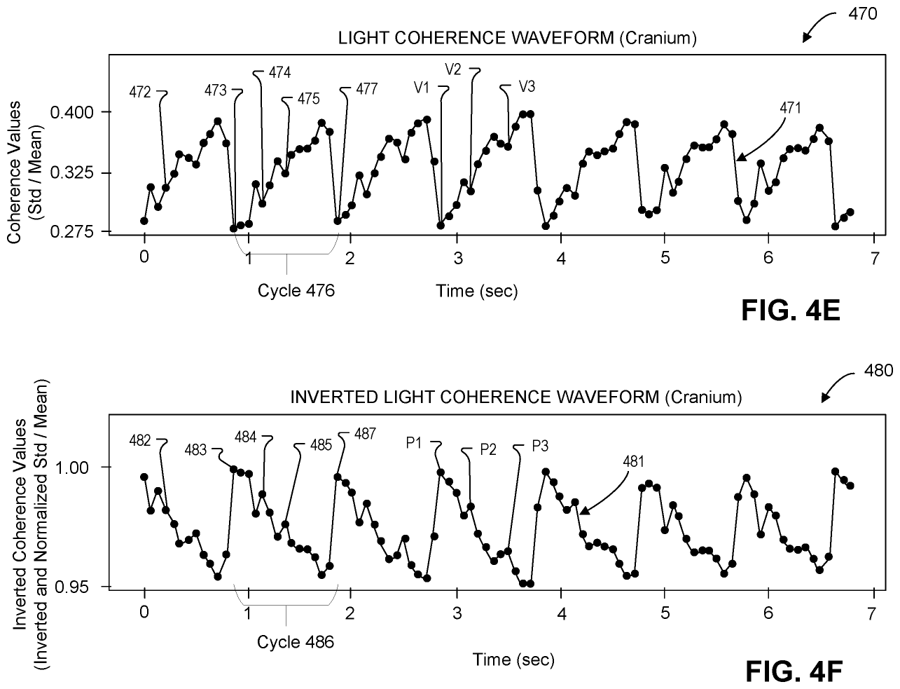

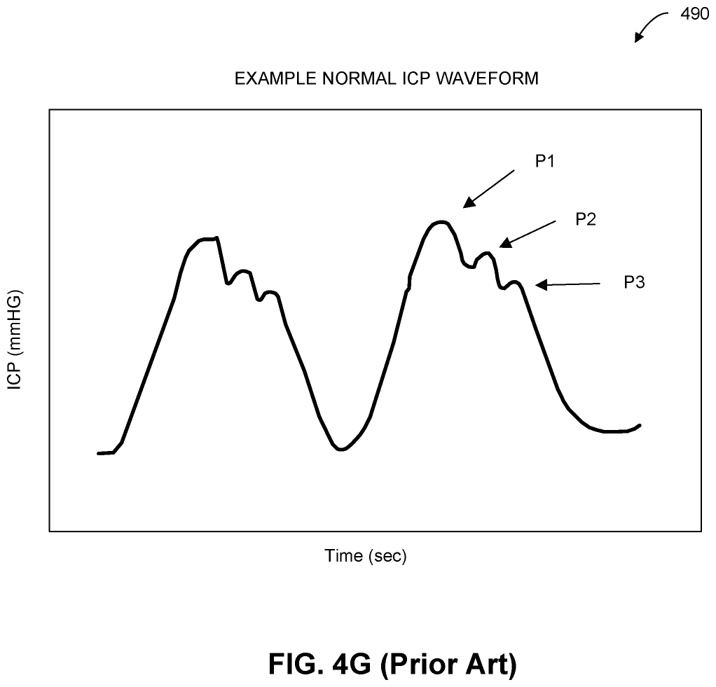

To capture motion, processing logic 108 is configured to sequentially capture images at time intervals. For example, processing logic 108 may be configured to capture 10, 20, 50, or 100 images per heartbeat or per second to define or provide resolution to the waveform of coherence values (e.g., shown in FIGS. 4E and/or 4F).

Processing logic 108 is configured to perform diffused light coherence analysis on image data 122 to identify intracranial motion characteristics within cranium 101, in an embodiment. Processing logic 108 may perform coherence analysis on image data 122 to define coherence values by performing one or more of the following operations: calculating the variance of the pixels of an image, calculating the standard deviation of the pixels of an image, calculating the mean of the pixels of the image, and/or defining a speckle contrast value as the standard deviation divided by the mean of the pixels of the image.

Coherence values may be used to provide intracranial motion characteristics in cranium 101 and/or tissue sample 102. In one embodiment, coherence values vary based on blood volume passing through blood vessels 124. Blood vessels 124 may include larger blood vessels 124A and smaller blood vessels 124B. Larger blood vessels 124A may include arterioles, metarterioles, thoroughfare channels, and venules. Smaller blood vessels 124B may include capillaries. Smaller blood vessels 124B may contribute more significantly to coherence values than larger blood vessels 124A. Coherence may be mapped or modeled to be inversely related to blood motion, blood vessel motion, brain tissue motion, and/or cranium motion. In one application, coherence could be inversely proportional to blood motion, blood vessel motion, brain tissue motion, and/or cranium motion. Coherence is inversely related to intracranial motion in that coherence decreases with increases in motion, e.g., increases in blood volume passing through blood vessels 124. Additionally, coherence increases with decreases in motion, e.g., decreases in blood volume passing through blood vessels 124. Coherence values may be compared to modeled intracranial motion characteristics to identify decreases in motion (e.g., blood flow volume) in order to identify the presence of blood clots or other vascular occlusions. As a result, embodiments of the disclosure may be used to characterize health issues (e.g., an ischemic or hemorrhagic stroke) associated with decreases in intracranial motion.

Processing logic 108 applies rules and/or data models to acquired coherence values or to coherence value waveforms to characterize blood flow performance within cranium 101. The rules may be based on various characteristics of the coherence value waveforms, such as, the number of peaks in a cycle of a waveform, the number of valleys in a cycle of the waveform, the expressions of blood flow cycles within a cycle of a waveform, the curvature of peaks in the waveform, etc. Data models may vary based on the size or age of tissue sample 102 and/or based on characteristics of the test subject. Coherence values within cranium 101 may differ for various parts of cranium 101 (e.g., front, rear, left, right, top, bottom, etc.). Coherence values within cranium 101 may differ based on characteristics of a test subject (e.g., body mass index “BMI”, gender, age, height, fitness level, genetics, health, cholesterol levels, etc.). Accordingly, processing logic 108 may receive characteristics of a test subject and compare coherence values against one or more particular data models (from a plurality of data models), to determine intracranial motion characteristics and intracranial blood flow performance. The aforementioned rules and data models are described in addition detail hereafter in association with FIGS. 4E, 4F, 5B, 9, and 10, for example.

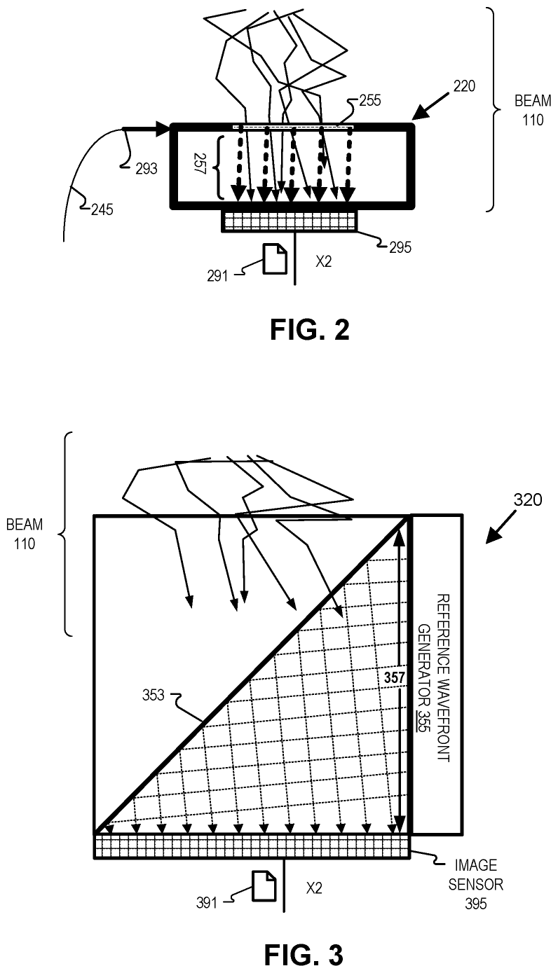

FIG. 2 illustrates a light detector 220 that is configured to capture coherent light interference patterns to determine intracranial motion characteristics, in accordance with aspects of the disclosure. Light detector 220 is an example implementation a portion of light detector 106 (shown in FIG. 1). Light detector 220 may include an image sensor 295 configured to image one or more interference patterns generated by a reference beam 257 interfering with measurement beam 110. Light detector 220 is configured to receive measurement beam 110. Reference beam 257 is the same wavelength as laser light emitted by light generator 114 of light source 104, in some embodiments. Reference beam 257 may be provided to an optical input 293 of light detector 220 by an optical fiber 245 that receives (for example) laser light from light generator 114, to ensure the wavelength of reference beam 257 is the same as the wavelength of measurement beam 110. In the illustration of FIG. 2, a reference optical element 255 is configured to direct reference beam 257 to illuminate image sensor 295. Reference optical element 255 may include a surface relief grating, Bragg grating, and/or a holographic optical element coupled to receive the reference beam from optical input 293 and direct reference beam 257 to image sensor 295. In some embodiments, reference optical element 255 is configured to direct reference beam 257 to become incident upon the image sensor 295 at an angle slightly offset from an angle that is perpendicular to an imaging plane of the image sensor 295.

Light detector 220 is configured to capture image data 291 of the interference pattern generated by measurement beam 110 interfering with reference beam 257. Processing logic 108 (shown in FIG. 1) may be configured to initiate the image capture by image sensor 295 via communication channel X2. The intensity of the interference pattern is measured by coherence values. The intensity of the interference pattern is captured by the image sensor 295 of light detector 220 can be analyzed using techniques disclosed herein to determine intracranial motion characteristics over time (e.g., blood flow rates, hemoglobin oxygenation levels, blood vessel displacement, brain tissue displacement, etc.). For example, the interference pattern from diffuse light and a reference beam may include fringes in image data 291, and the contrast between fringe patterns in image data 291 may be used to determine intracranial motion characteristics (e.g., blood flow characteristics).

FIG. 3 illustrates a light detector 320 that is configured to capture coherent light interference patterns to determine intracranial motion characteristics and blood flow performance within a cranium, in accordance with aspects of the disclosure. Light detector 320 is an example implementation of light detector 106 (shown in FIG. 1). Light detector 320 is configured to receive measurement beam 110. Light detector 320 includes an image sensor 395 configured to capture image data 391 of an interference between measurement beam 110 and reference beam 357. At least a portion of measurement beam 110 propagates through beam splitter 353 to interfere with the portion of reference beam 357 that is reflected back toward image sensor 395. Therefore, image data 391 generated by image sensor 395 is representative of an interference of reference beam 357 with measurement beam 110.

Reference wavefront generator 355 generates reference beam 357, which may be a near-infrared reference beam or a visible light reference beam. Reference wavefront generator 355 may include one or more lasers and corresponding optics to generate a substantially uniform wavefront for reference beam 357. Reference wavefront generator 355 may receive light from a same light generator (e.g., light generator 114 shown in FIG. 1) that provides light for light source 104, in some embodiments. Reference beam 357 may be the same wavelength, phase, and/or polarity as the light emitted from light source 104. Or, reference beam 357 may be the same wavelength as a (doppler) wavelength shifted portion of measurement beam 110, in some implementations.

In one embodiment, reference wavefront generator 355 is disposed to effect delivery of the reference beam 357 to image sensor 395 at an angle to a pixel plane of the image sensor 395. Image sensor 395 may include image pixels disposed in two-dimensional rows and columns that define the pixel plane of the image sensor 395. Processing logic 108 may be configured to initiate the image capture by image sensor 395 via communication channel X2.

FIGS. 4A, 4B, 4C, 4D, 4E, and 4F illustrate graphical representations of intracranial motion characteristics (including blood characteristics) that may be incorporated into one or more data models and/or rule sets to determine intracranial blood flow performance from coherent light interference patterns, in accordance with embodiments of the disclosure.

FIG. 4A illustrates a speckle contrast graph 400 that demonstrates how speckle contrast values for image data (e.g., image data 122 of FIG. 1, image data 291 of FIG. 2, image data 391 of FIG. 3, etc.) may vary based on intracranial motion characteristics (e.g., blood flow), in accordance with embodiments of the disclosure. Speckle contrast graph 400 includes an x-axis and a y-axis. The x-axis includes time in seconds, and the y-axis includes speckle contrast values, which may be defined as the standard deviation of (e.g., all or a sub-set of) pixel values of an image divided by the mean of (e.g., all or a sub-section of) pixel values of the image. Speckle contrast graph 400 includes a data line 402 that shows an example of speckle contrast values varying with respect to time when blood flow (or simulated blood flow) is at least partially constricted, released, and at least partially constricted. Data line 402 represents measurements captured by a light detector positioned 10 mm from a light source. Data line 404 is similar to data line 402, but data line 404 represents measurements captured by a light detector positioned 20 mm from a light source. Data line 406 is similar to data line 402, but data line 406 represents measurements captured by a light detector positioned 30 mm from a light source. Speckle contrast values decrease when blood flow (or other motion) increases because coherent light interference decreases with motion. Speckle contrast values decrease with blood flow increases, while a tissue sample is illuminated with light having wavelengths in the range of approximately 600 nm to 1000 nm, according to embodiments of the disclosure. Speckle contrast values vary with changes in distance between a light source and a light detector, and the speckle contrast values vary with changes in exposure time of the tissue sample to coherent light.

Although speckle contrast graph 400 is described in terms of speckle contrast, speckle contrast is but one example of a coherence value that may be used to quantify coherent light interference patterns, in accordance with aspects of the disclosure. Other examples of coherence values that may be used include the variance of pixels in an image and the standard deviation of pixels in an image. Thus, although speckle contrast is specifically identified, it is to be understood that coherence values may include variance values, standard deviation values, and/or speckle contrast values, according to various implementations. Additionally, as described below in association with FIG. 4F, a sequentially acquired waveform of coherence values may be inverted (e.g., flipped upside down) and evaluated as a graph that resembles an intracranial pressure (“ICP”) waveform, according to an embodiment of the disclosure.

FIG. 4B illustrates a blood flow index graph 420 that demonstrates how a data model may map speckle contrast values against distance for a particular blood flow index value, in accordance with embodiments of the disclosure. Blood flow index graph 420 includes an x-axis and a y-axis. The x-axis includes distance between a light source and light detector in millimeters, and the y-axis includes speckle contrast values. Blood flow index graph 420 includes a data line 422 that models illustrative speckle contrast values, as they change with distance between a light source and a light detector. Data line 422 is an example of values that may be modeled for a particular blood flow index, e.g., 5.88×10⁻⁶ mm²/s. Data points 424A, 424B, and 424C represent test measurements made at 10 mm, 20 mm, and 30 mm that fit relatively well to an example data model. The data line 422 is non-linear, indicating that a non-linear relationship exists between speckle contrast values and distance between source and detector optical fibers, according to an embodiment.

FIG. 4C illustrates a blood flow index graph 440 that demonstrates how blood flow index values may vary based on intracranial motion characteristics (e.g., blood characteristics), with respect to time, in accordance with embodiments of the disclosure. Blood flow index graph 440 includes an x-axis and a y-axis. The x-axis includes time in seconds, and the y-axis includes blood flow index values in squared millimeters per seconds (mm²/s). The blood flow index values represent a quantity of blood flowing through a two-dimensional cross-section per second, without regard to the diameter of the blood vessel. The blood flow index may be determined by multiplying a fraction of scatterers that are moving (e.g., blood cells and/or hemoglobin) by a diffusion coefficient of the scatterers. The fraction of scatterers that are moving are represented as “alpha”, and the diffusion coefficient of the scatterers is represented as “Db” in the y-axis descriptor. The diffusion coefficient may be an effective Brownian diffusion coefficient used to model scatterers undergoing Brownian motion. Blood flow index graph 440 includes a data line 442 that illustrates an increase in blood flow, for example, from 5.25×10⁻⁶ mm²/s to 6.75×10⁻⁶ mm²/s, in response to de-constricting blood vessels (e.g., at time 40 s). Blood flow index graph 440 includes a data line 442 that illustrates a decrease in blood flow index, for example, from 6.75×10⁻⁶ mm²/s to 5.4×10⁻⁶ mm²/s, in response to constricting blood vessels (e.g., at time 90 s).

FIG. 4D illustrates a blood flow index graph 460 that demonstrates how a data model may map blood flow index values against blood flow speeds, in accordance with embodiments of the disclosure. The x-axis includes blood flow speeds in millimeters per second. The y-axis includes blood flow index values in squared millimeters per second, scaled by 10⁻⁶ (i.e., 10⁻⁶ mm²/s). Blood flow index graph 460 includes a data line 462 that shows an example of data that models a map of blood flow index values against blood flow speed. Data line 462 may model measurements of a 4 mm diameter blood vessel located approximately 15 mm from the skin surface, as an example. Blood flow index graph 460 and the graphs of FIGS. 4A-4C are illustrative examples of what data may look like and are representative of how coherence values, such as speckle contrast, may be modeled and may be representative of various blood characteristics.

Other blood characteristics may be modeled, measured, and used to obtain information about blood flow within a tissue sample. For example, the mean value of an image may be determined for each image to quantify an intensity of an image. The intensity of captured images may be used to generate a data model of intensity versus distance between a light source and a light detector (e.g., in millimeters). The data model may be built to include values for a variety of optical attenuation coefficients, which may be represented as μ or μ_eff. The units of an optical attenuation coefficient (μ_eff) may be mm⁻¹ or per millimeter. The optical attenuation coefficient may be captured over time and may have different values when blood flow is constricted (e.g., via a clot or other occlusion) versus free flowing. In an embodiment, optical attenuation coefficient is determined to classify blood characteristics in a tissue sample.

FIG. 4E illustrates a light coherence graph 470 of coherence values sequentially captured at time intervals to represent intracranial motion characteristics, in accordance with an embodiment of the disclosure. Light coherence graph 470 includes an x-axis of time (in seconds) and a y-axis of coherence values. Coherence values can include mean pixel values of an image, a variance of pixel values of an image, a standard deviation of pixel values of an image, or speckle contrast (standard deviation divided by mean) of pixel values of an image. The coherence values of light coherence graph 470 illustrate coherence values that are speckle contrast values, for illustrative purposes.

Light coherence graph 470 is a waveform that includes a number of characteristics that represent intracranial motion and that may be used to identify reduced intracranial blood flow. Light coherence graph 470 includes a waveform 471 that is constructed from a number of data points 472. Each of data points 472 represents a coherence value of coherent light interference patterns captured by an image of pixels by a light detector (e.g., light detector 106 shown in FIG. 1) from a cranium (e.g., cranium 101 shown in FIG. 1). Waveform 471 includes a first valley 473, a second valley 474, and a third valley 475 within a cycle 476 in waveform 471. Cycle 476 spans from first valley 473 to valley 477. Valley 477 represents a first valley of a cycle that is subsequent to cycle 476. Generically, each cycle of waveform 471 may include three valleys, which are labeled V1 (first valley), V2 (second valley), and V3 (third valley) in a fourth cycle of waveform 471. A cycle may be defined as a spatial representation of a repeating pattern of coherence values with respect to time within a waveform. The relative values of the valleys, the omission of valleys, and the cycle shape relative to cycle shapes captured from different locations on a cranium may all be characteristics of waveform 471 that may be used to identify potential reduction of intracranial blood flow.

FIG. 4F illustrates an inverted light coherence graph 480 of inverted and normalized coherence values sequentially captured at time intervals to represent intracranial motion characteristics, in accordance with an embodiment of the disclosure. Inverted light coherence graph 480 includes an x-axis of time (in seconds) and a y-axis of inverted and normalized coherence values. The coherence values of inverted light coherence graph 480 illustrate inverted coherence values that are speckle contrast values, for illustrative purposes. Other coherence values may alternatively be employed.

Inverted light coherence graph 480 is a waveform that resembles an intracranial pressure (“ICP”) waveform. Inverted light coherence graph 480 includes a number of characteristics that represent intracranial motion and that may be used to identify reduced intracranial blood flow or other intracranial blood flow performance metrics. Inverted light coherence graph 480 includes a waveform 481 that is constructed from a number of data points 482. Each of data points 482 represents an inverted coherence value of coherent light interference patterns captured by an image of pixels by a light detector (e.g., light detector 106 shown in FIG. 1) from a cranium (e.g., cranium 101 shown in FIG. 1). Inversion of the coherence values may be performed a number of ways, such as mirroring the value across the x-axis and then normalizing the values to be on the positive y-axis (graphically). Waveform 481 includes a first peak 483, a second peak 484, and a third peak 485 within a cycle 486 in waveform 481. Cycle 486 spans from first peak 483 to peak 487. Peak 487 represents a first peak of a cycle that is subsequent to cycle 486. Generically, each cycle of waveform 481 may include three peaks, which are labeled P1 (first peak), P2 (second peak), and P3 (third peak) in another cycle. The relative values of the peaks, the omission of peaks, and the cycle shape relative to cycle shapes captured from different locations on a cranium may all be characteristics of waveform 481 that may be used to identify potential reduction of intracranial blood flow. Since waveform 481 resembles an ICP waveform, P1, P2, and P3 may be analyzed as though they were a first, second, or third ICP waveform peak. In other words, P1, P2, and P3 of waveform 481 may be analyzed as though they were a P1 percussion wave, a P2 tidal wave, and a P3 dicrotic wave of an ICP waveform to characterize intracranial motion and to potentially identify reduction of intracranial blood flow, as is symptomatic of some types of strokes.

FIG. 4G illustrates an example normal ICP waveform 490 as is currently captured by the medical profession using a transducer placed within a subject's cranium through a bored aperture in the subject's cranium. Example normal ICP waveform 490 includes an x-axis of time (in seconds) and a y-axis of ICP values measured in millimeters of Mercury. Example normal ICP waveform 490 includes three peaks P1, P2, and P3, which represent a percussion wave, a tidal wave, and a dicrotic wave of an ICP waveform, respectively.

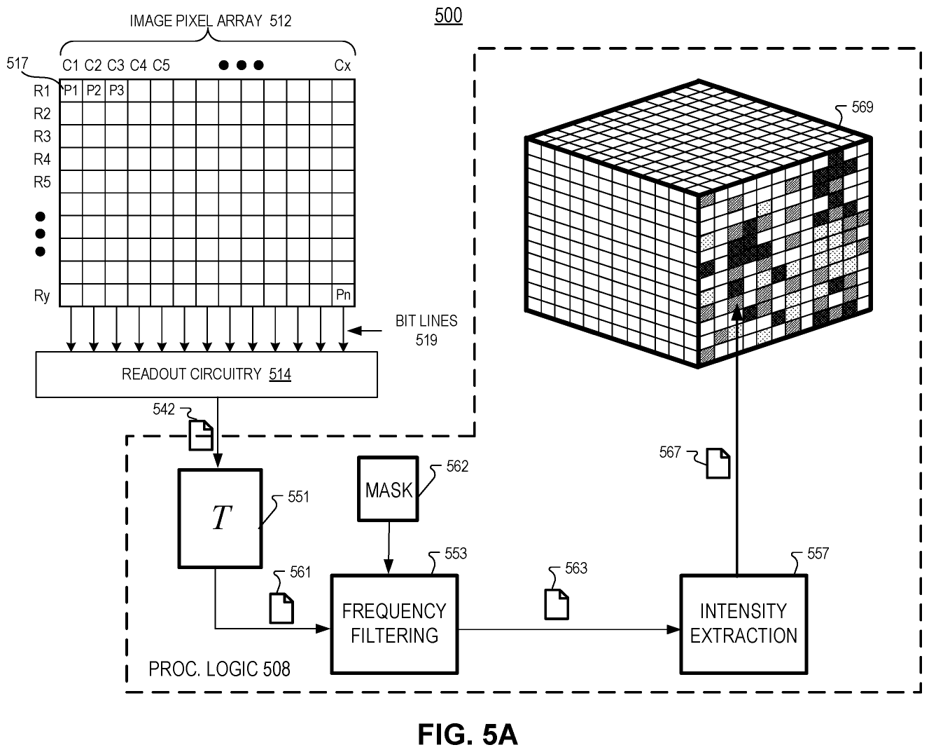

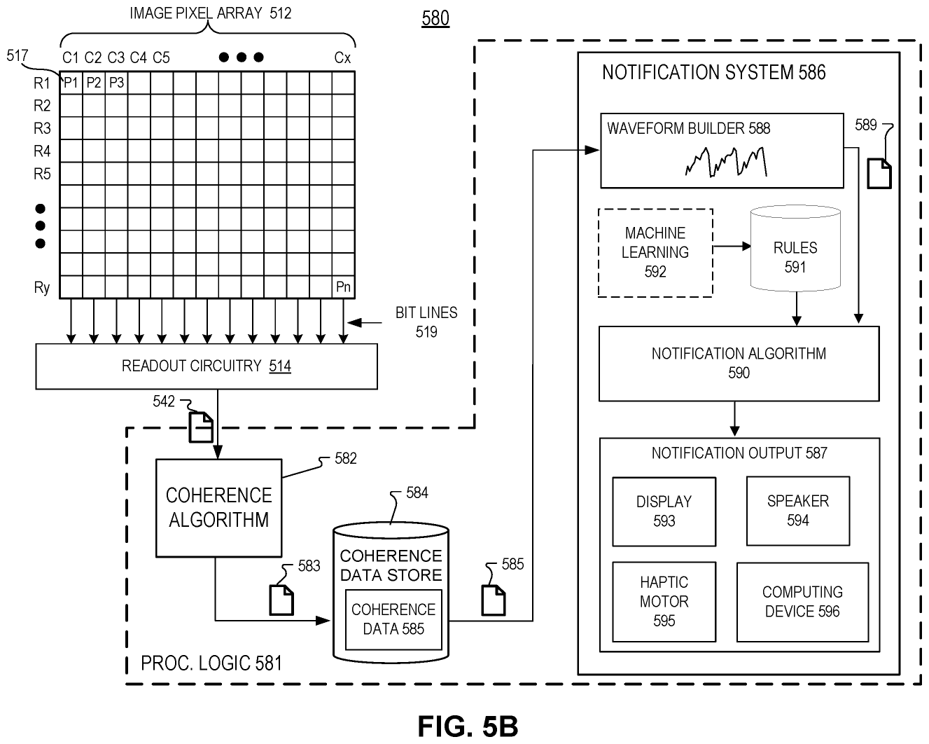

FIG. 5A illustrates a processing logic system 500 that may be an implementation of processing logic (e.g., processing logic 108 of FIG. 1) to process image data (e.g., image data 122 of FIG. 1, image data 291 of FIG. 2, image data 391 of FIG. 3, etc.) from an image sensor (e.g., image sensor 118 of FIG. 1, image sensor 295 of FIG. 2, image data 391 of FIG. 3, etc.), in accordance with an embodiment of the disclosure. Processing logic system 500 includes an image pixel array 512 coupled to processing logic 508. Image pixel array 512 represents a pixel array that may be included in an image sensor (e.g., image sensor 118, 295, 395). Processing logic 508 includes features that may be included in processing logic 108, according to an embodiment of the disclosure. Image pixel array 512 includes image pixels 517 arranged in integer number x columns (C1-Cx) and integer number y rows (R1-Ry). Readout circuitry 514 is coupled to read the signal value from each image pixel 517 via bitlines 519. Transform engine 551 in processing logic 508 is coupled to receive image 542 from readout circuitry 514. Image 542 may be an example of image data 122. Transform engine 551 generates a frequency domain image 561 by performing a Transform operation on image 542 received from readout circuitry 514. In one embodiment, the Transform operation includes an inverse Fourier transform. In one embodiment, the Transform operation includes a discrete cosine transform.

Frequency filtering engine 553 is coupled to receive the frequency domain image 561 from Transform engine 551 and also coupled to receive mask 562. Frequency filtering engine 553 is configured to multiply the frequency domain image 561 with the mask 562 to generate a filtered frequency domain image 563, in the illustrated embodiment of FIG. 5A. Mask 562 is designed to isolate the interference signal between the sample and reference light beams. Mask 562 may include a matrix that includes ‘1’ values for the portion of the frequency domain image 561 that corresponds to the interference of measurement beam 110 with the reference beam, and ‘0’ values for background signal in the frequency domain image 561. In one embodiment, mask 562 is a two-dimensional Gaussian filter.

Intensity extraction engine 557 is coupled to receive the filtered frequency domain image 563 and configured to extract intensity data 567 from the filtered frequency domain image 563. In one embodiment, generating the intensity data 567 includes averaging intensity values of the filtered frequency domain image 563. In an embodiment where a Fourier transform is used as the transform operation in Transform engine 551, the Fourier coefficients are extracted from filtered frequency domain image 563 and a sum of the logarithm of the absolute value of the Fourier coefficients is calculated. The sum is then used as intensity data 567. In some implementations, intensity extraction engine 557 may compare the sum of the logarithm of the absolute value of the Fourier coefficients to a baseline interference pattern in a baseline image of measurement beam 110 incident on image pixel array 512 that is captured without a tissue sample present to generate intensity data 567. In an embodiment, a baseline intensity value is subtracted from the sum of the logarithm of the absolute value of the Fourier coefficients of filtered frequency domain image 563 to generate intensity data 567 as a voxel value of composite image 569 for a particular measurement.

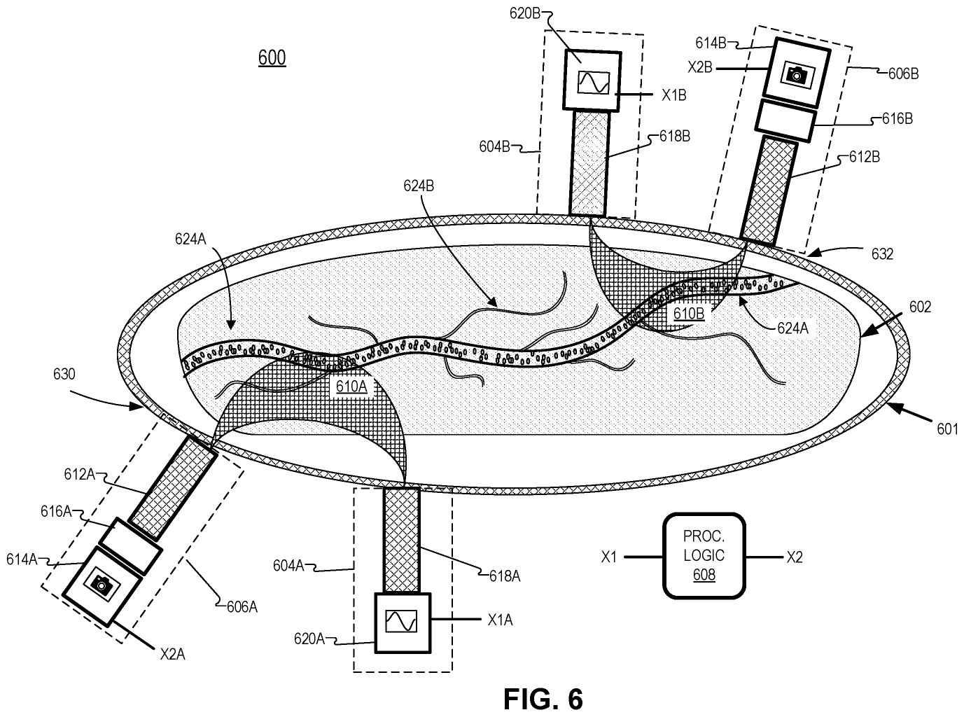

Processing logic 508 incorporates the intensity data 567 as a voxel value in a composite image 569. Composite image 569 is illustrated as a three-dimensional image in FIG. 5A and may be a three-dimensional image of a diffuse medium such as tissue sample 102 (shown in FIG. 1). In one embodiment, an imaging system (e.g., image system 100 of FIG. 1, imaging system 600 of FIG. 6, etc.) may employ a network of light sources and light detectors to gather motion and blood characteristics from various locations and depths within a cranium (or tissue sample) to generate a 3D composite image of a diffuse medium by generating a plurality of image data that correspond to the different voxels of the cranium.

FIG. 5B illustrates a processing logic system 580 that may be an implementation of processing logic 108 (shown in FIG. 1), to generate notifications based on intracranial motion data from an image sensor (e.g., image sensor 118 of FIG. 1), in accordance with an embodiment of the disclosure. Processing logic system 580 includes image pixel array 512 (shown in FIG. 5A) coupled to processing logic 581. Processing logic 581 includes features that may be included in processing logic 108 to determine intracranial motion characteristics (e.g., intracranial blood flow performance) from coherent light interference patterns. Processing logic 581 also includes features that may be included in processing logic 108 to generate notifications based on the intracranial motion characteristics, according to an embodiment of the disclosure.

Processing logic 581 may include a coherence algorithm 582 and a notification system 586 for determining intracranial motion characteristics from image 542, according to an embodiment of the disclosure. Coherence algorithm 582 is configured to quantify coherent light interference patterns from image 542 received by processing logic 581. Coherence algorithm 582 may be configured to determine coherence values for image 542 by calculating a variance, a standard deviation, and/or a speckle contrast of pixels values of image 542. In one implementation, coherence algorithm 582 calculates speckle contrast values using the standard deviation of pixel values divided by the mean of the pixel values in image 542. A number of operations may be incorporated into the coherence calculations, including, normalized electric field auto-correlation function, Gaussian moment theorem, pixel size, polarization purity, exposure time, power spectral density, and light bandwidth. Coherence algorithm 582 may generate a coherence value 583 for each image 542.

Processing logic 581 may store coherence value 583 in coherence data store 584. Coherence data store 584 may store each coherence value 583 as coherence data 585 when coherence value is combined with one or more of: a corresponding or captured time interval or time stamp, a location on a cranium where image 542 is captured, a distance between a light source and light detector used to generate image 542, a wavelength of a measurement beam, and/or information to identify a test subject. Coherence data store 584 is an array, table, database, or other data structure configured to store and associate a number of coherence values with corresponding interval times (time stamps) as coherence data 585. Processing logic 581 may transfer coherence data 585 to notification system 586 for further processing.

Notification system 586 is configured to receive coherence data 585 and generate a notification output 587, in response to analysis performed on coherence data 585, according to an embodiment. Notification system 586 analyzes features of coherence data 585 in terms of valleys, peaks, relative values of valleys/peaks, and/or the shape the waveform defined by coherence data 585. Notification system 586 may include a waveform builder 588 and a notification algorithm 590 that is used to generate notification output 587.

Waveform builder 588 reformats coherence data 585 into formatted coherence data 589 to facilitate generation of notification output 587. Waveform builder 588 may generate a visual representation of coherence data 585, so that coherence data 585 may be visualized as a waveform. Waveform builder 588 may add or remove data points to coherence data 585 to increase or decrease resolution to provide faster processing results or to improve one or more data processing techniques. Waveform builder 588 may be configured to invert and/or normalize coherence data 585 so that coherence values may be analyzed as inverted coherence values that may closely resemble ICP waveform data points. Waveform builder 588 may provide formatted coherence data 589 to notification algorithm 590 as both coherence values and inverted coherence values that are associated with a relative or absolute time stamp.

Notification system 586 includes rules 591 that define coherence waveform and inverted coherence waveform characteristics that may indicate reduced intracranial blood flow. Rules 591 for determining reduced intracranial blood flow may be manually entered or may be defined by applying machine learning engine 592 (e.g., neural networks, regression, classification, etc.) to tens, hundreds, thousands, or more samples of coherence waveforms. Machine learning engine 592 or processing logic 581 may be configured to apply independent component analysis (“ICA”), artificial neural network models, wavelet analysis, or other data or waveform analysis techniques to characterize or extract information from waveforms. Rules 591 may include data models of ideal, normal, unknown, or reduced intracranial blood flow.

A first example rule that may be included in rules 591 relates to relative valley values in a cycle of a light coherence waveform. The first example rule may be defined as: a minimum coherence value of a second valley (e.g., second valley 474 of FIG. 4E) is equal to or less than a minimum coherence value of a first valley (e.g., first valley 473 of FIG. 4E). If this first rule is true, then the light coherence waveform (or a cycle therein) may indicate that the potential for reduced intracranial blood flow exists in a test subject.

A second example rule that may be included in rules 591 relates to the shape of a cycle of a light coherence waveform. The second example rule may be defined as: an absence of a second valley (e.g., second valley 474 of FIG. 4E) and of a third valley (e.g., third valley 475 of FIG. 4E), such that a single valley and a single peak exist within a cycle. Such a characteristic may be indicative of a lack of a tidal wave and dicrotic wave (in an ICP waveform), which may indicate high intracranial pressure. If this second rule is true, then the light coherence waveform (or a cycle therein) may indicate that the potential for reduced intracranial blood flow exists in a test subject.

A third example rule that may be included in rules 591 relates to the relative shapes of cycles of light coherence waveforms measured from different cranial locations. The third example rule may be defined as: relative valley values of a cycle of a waveform captured from a first location on a cranium differs from relative valley values of a cycle of a waveform captured concurrently from a second location on a cranium. If this third rule is true, then the light coherence waveform (or a cycle therein) may indicate that the potential for reduced intracranial blood flow exists in a test subject.

A fourth example rule that may be included in rules 591 relates to relative peak values in a cycle of an inverted light coherence waveform (e.g., waveform 481 shown in FIG. 4F). The fourth example rule may be defined as: a maximum coherence value of a second peak (e.g., second peak 484 of FIG. 4F) is equal to or greater than a maximum coherence value of a first peak (e.g., first peak 483 of FIG. 4F). This rule may mimic rules applied to ICP waveforms generated using intracranially disposed transducers. If this fourth rule is true, then the light coherence waveform (or a cycle therein) may indicate that the potential for reduced intracranial blood flow exists in a test subject.

A fifth example rule that may be included in rules 591 relates to the shape of a cycle of an inverted light coherence waveform. The fifth example rule may be defined as: an absent of a second peak (e.g., second peak 484 of FIG. 4F) and of a third peak (e.g., third peak 485 of FIG. 4F), such that a single valley and a single peak exist within a cycle (e.g., cycle 486 of FIG. 4F). Such a characteristic may be indicative of a lack of a tidal wave and dicrotic wave (in an ICP waveform), which may indicate high intracranial pressure. If this fifth rule is true, then the light coherence waveform (or a cycle therein) may indicate that the potential for reduced intracranial blood flow exists in a test subject.

Notification algorithm 590 applies one or more rules 591 to formatted coherence data 589 (or to coherence data 585) received from waveform builder 588. Notification algorithm 590 may apply rules 591 to coherence values that represent coherence waveforms and to formatted coherence values that represent inverted coherence waveforms. Notification algorithm 590 may be configured to generate a variety of notification outputs based on rules 591. In one embodiment, notification algorithm 590 outputs a first output indicating that the intracranial blood flow performance is unknown. In one embodiment, notification algorithm 590 outputs a second output indicating that the intracranial blood flow performance is potentially reduced. Notification algorithm 590 may be configured to deliver more or fewer notification messages.

Notification output 587 is configured to notify a person (e.g., medical staff) of when intracranial motion characteristics indicate that a test subject has potentially reduced intracranial blood flow. Notification system 586 provides notification output 587 to a display 593, a speaker 594, a haptic motor 595, and/or a computing device 596 (e.g., e-mail, text message, electronic file, etc.).

FIG. 6 illustrates an imaging system 600 that includes a network of light detectors and light sources to perform comparative intracranial motion analysis and/or to generate data for composite images (e.g., images 569 FIG. 5A), in accordance with embodiments of the disclosure. Imaging system 600 is applied to a cranium 601 and a brain tissue sample 602 and includes a number of light sources 604 (individually, light sources 604A and 604B), a number of light detectors 606 (individually, light detectors 606A and 606B), and processing logic 608. Brain tissue sample 602 may include the features of tissue sample 102 of FIG. 1, each of light sources 604 may include the features of light source 104 of FIG. 1, each of light detectors 606 may include the features of light detector 106 of FIG. 1, and processing logic 608 may include the features of processing logic 108 of FIG. 1, in an embodiment.

As illustrated, imaging system 600 may have light detectors 606 distributed in various locations around cranium 601 to determine intracranial motion characteristics from a variety of locations within cranium 601 and/or brain tissue sample 602. Each of light detectors 606 may be controlled by and communicate with processing logic 608 over communications channels X2A and X2B (collectively, communications channels X2). Light detectors 606 capture light and images of measurement beams 610A-B, for example. Light detector 606A is positioned on a first side 630 of cranium 601 and light detector 606B is positioned on a second side 632 of cranium 601 to enable processing logic 608 to perform a comparative analysis of intracranial motion characteristics of blood vessels 624 within brain tissue sample 602. Blood vessels 624 may include larger blood vessels 624A (e.g., arterioles, metarterioles, thoroughfare channels, and venules) and smaller blood vessels 624B (e.g., capillaries).

Light detectors 606 may include optical fibers 612A-B, image sensor 614A-B (e.g., CMOS, CCD, etc.), and optical converters 616A-B (e.g., optical switch, lens, etc.).

Each of light sources 604 may include an optical fiber 618, and a light generator 620. Optical fiber 618 may be a multi-mode optical fiber having a core diameter of 50 μm, 62.5 μm, or some other diameter that is greater than 10 μm. In some implementations, optical fiber 618 is a multi-modal optical fiber having a core diameter of 1 mm or greater. Light generator 620 may be a continuous wave laser that is selectively chopped or operated to provide predetermined durations of illumination within brain tissue sample 602. Each of light sources 604 may be controlled by and communicate with processing logic 608 over communications channels X1A and X1B (collectively communications channels X1). Imaging system 600 may be implemented with a single light source 604A and may be implemented with one or more additional light sources, such as light source 604B. Light source 604B may use the same light generator 620A as light source 604A or may have a different light generator 620B. Light generator 620B may be a different wavelength of light than the wavelength of light generator 620A, in an embodiment.

To facilitate a comparative analysis of light coherence waveforms captured from two or more locations on cranium 601, a distance between light source 604A and light detector 606A may be set to be the same as a distance between light source 604B and light detector 606B. The depth of measurement achieved by measurement waves 610 at least partially depends on a distance between a light source and a light detector. To target specific areas within cranium 601 and/or brain tissue sample 602, distances between light source 604 and light detector 606 may be lengthened (e.g., to 30 mm or greater) or may be decreased (e.g., to 10 mm or shorter) depending upon the specific location of interest.

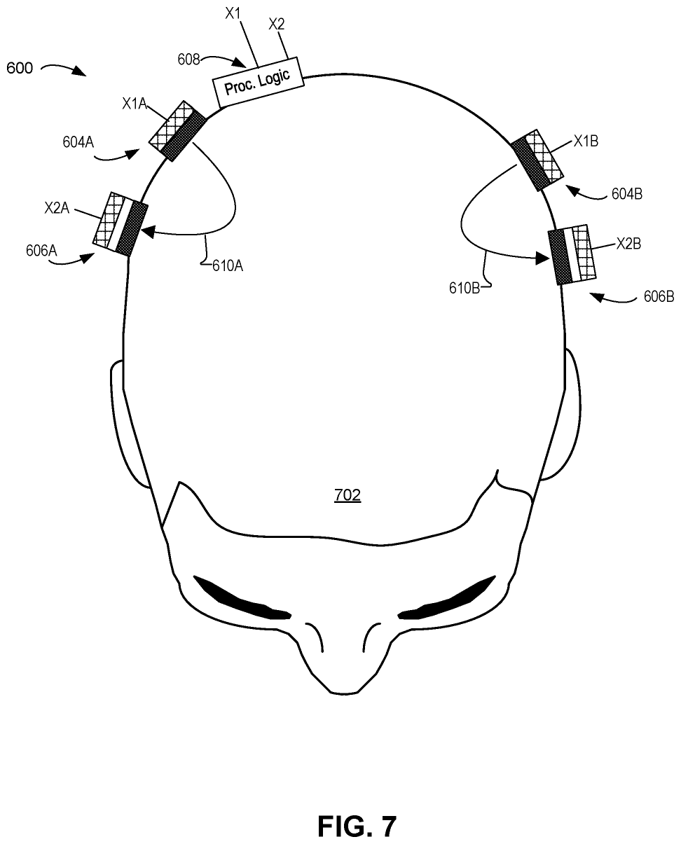

FIG. 7 illustrates an example placement of components of imaging system 600 (shown in FIG. 6) in relationship to a human head, in accordance with an embodiment of the disclosure. FIG. 7 is a top-down view of a human head 702. Light sources 604A-B may be positioned to provide light that is diffused within human head 702. Portions of the diffused light, such as measurement beams 610A-B, may be captured by light detectors 606A-B at a variety of locations around human head 702. A wearable hat or other sensor carrying device may include system imaging system 600 can be worn as a wearable, in some embodiments. Other wearables may also include all or part of imaging system 600.

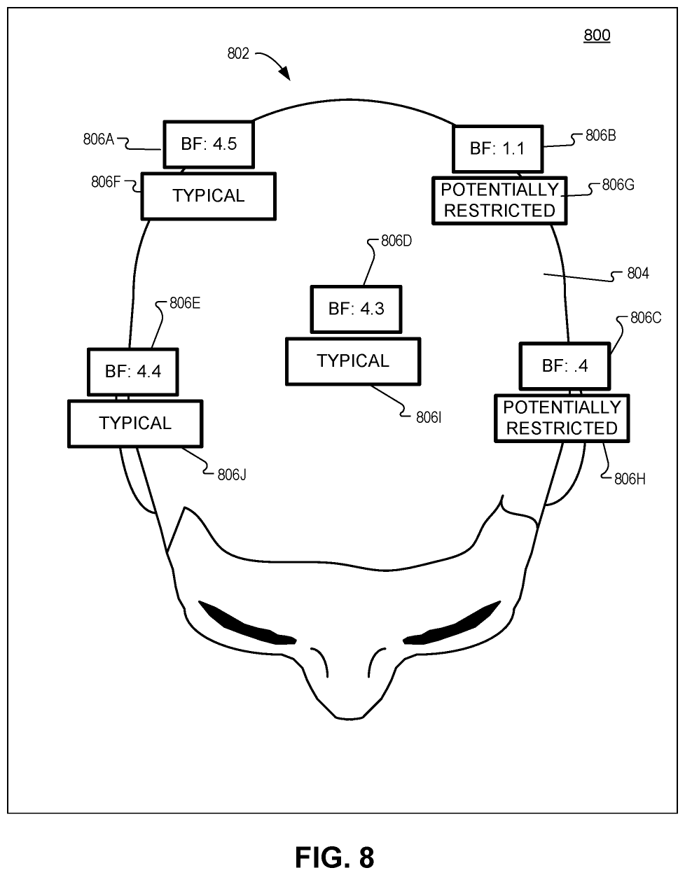

FIG. 8 illustrates an example display 800 that includes a composite image 802 of a human head 804, to provide easily viewable/readable intracranial blood flow performance, in accordance with an embodiment of the disclosure. Composite image 802 may include a number of display values 806 (individually, 806A, 806B, 806C, 806D, 806E, 806F, 806G, 806H, 806I, 806J). Display values 806A-E may be configured to display blood flow (BF) indices, rates, or volume. Display values 806F-J may be configured to display a text representation of blood flow performance as, for example, potentially restricted, typical, nominal, unknown, or the like, to provide an at-a-glance update to a viewer. Example display 800 may also include flashing text or color changes (e.g., from green to red) to draw attention to a blood flow performance status of potentially restricted, for example.

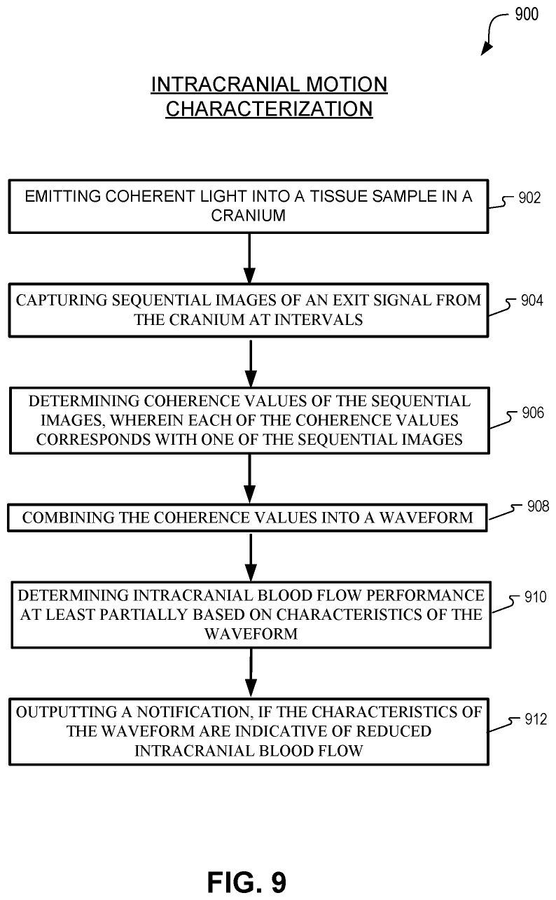

FIG. 9 illustrates a process 900 for determining intracranial motion characteristics from coherent light interference patterns, in accordance with an embodiment of the disclosure. The operations of process 900 may be performed in the order described or in another order, according to various embodiments.

At operation 902, process 900 includes emitting coherent light into a tissue sample in a cranium, according to an embodiment. An example of coherent light includes laser light where the emitted radiation includes waves vibrating in the same phase, same amplitude, polarity, and same wavelength. The laser light is emitted with wavelengths of 600-900 nm, in an embodiment. The laser light is configured to be emitted at 850 nm, in an embodiment. The laser light is provided with a pulse duration including the range of 10 μs to 100 μs, in an embodiment. The laser light is provided at one or more of multiple different pulses widths, including 10 μs, 20 μs, 40 μs, and 80 μs, in an embodiment.

At operation 904, process 900 includes capturing sequential images of an exit signal from the cranium at intervals, according to an embodiment. The one or more detector optical fibers are multi-mode optical fibers, for example, having a core diameter that is greater than 10 μm. Examples of multi-mode optical fiber include (e.g., glass or plastic) optical fibers having a core diameter of 50 μm, 62.5 μm, 200 μm, 1 mm, or the like. In one embodiment, the one or more detector optical fibers are single-mode optical fibers, for example, having a core diameter of 9 μm or less. Alternatively, other types of optical elements (e.g., a small window, a system of lenses, etc.) may be positioned between the cranium at the image sensor, instead of optical fiber, to facilitate transmission of the exit signal between the cranium and the image sensor. The image sensor may be a CMOS or CCD image sensor.

At operation 906, process 900 includes determining coherence values of the sequential images, wherein each of the coherence values corresponds with one of the sequential images, according to an embodiment. Coherent light interference patterns are analyzed by determining a coherence value for each image. A coherence value may be a variance, a standard deviation, and/or speckle contrast of the image. Speckle contrast may be defined, for example, as dividing the standard deviation of the pixels of the image by the mean of the pixels of the image, in an embodiment.

At operation 908, process 900 includes combining the coherence values into a waveform, according to an embodiment. The waveform represents motion within a cranium. The waveform may be saved in a table, chart, database, or other data structure and may include coherence values associated with respective time intervals (or time stamps). The waveform data may also include an indication of which light detector, light source, cranial location, wavelength, and distance between light source and light detector was used to generate the waveform. Lower coherence values represent more motion within the cranium, and higher coherence values represent less motion within the cranium. Motion may be attributed to blood cell motion, blood vessel motion, brain tissue motion, and/or cranial vibration.

At operation 910, process 900 includes determining intracranial blood flow performance at least partially based on characteristics of the waveform, according to an embodiment. Process 900 may apply one or more of a number of rules (e.g., rules 591 of FIG. 5B) to the waveform data to determine if a cycle within the waveform represents reduced intracranial blood flow.

At operation 912, process 900 includes outputting a notification, if the characteristics of the waveform are indicative of reduced intracranial blood flow, according to an embodiment. In one embodiment, the characteristics of the waveform are indicative of reduced intracranial blood flow if the probability or likelihood of reduced intracranial blood flow is at least 50%, or more likely than not. In another embodiment, the characteristics of the waveform are indicative of reduced intracranial blood flow if the probability or likelihood of reduced intracranial blood flow is a 30% probability, a 60% probability, or some other pre-determined or user-set likelihood upon which notification is desired. A notification may be output to a display, a speaker, a haptic motor, or to a computing device (e.g., via email, a file, etc.). The notification may include numerical data (e.g., blood flow rates, a blood flow index) and/or may include text-based messages (e.g., nominal blood flow, typical blood flow, unknown blood flow, and/or reduced blood flow), for example.

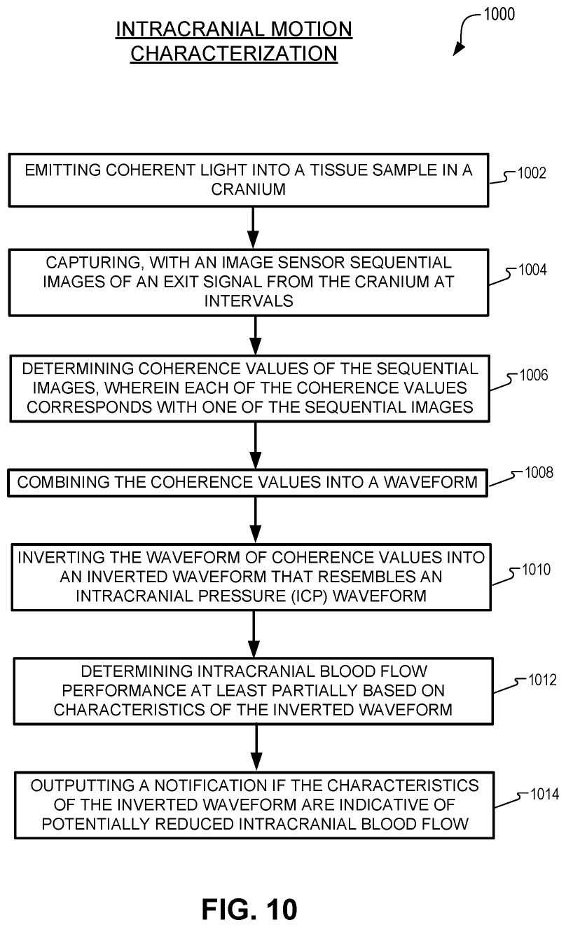

FIG. 10 illustrates a process 1000 for determining intracranial motion characteristics from coherent light interference patterns, in accordance with an embodiment of the disclosure. The operations of process 1000 may be performed in the order described or in another order, according to various embodiments.

At operation 1002, process 1000 includes emitting coherent light into a tissue sample in a cranium, according to an embodiment. An example of coherent light includes laser light where the emitted radiation includes waves vibrating in the same phase, same amplitude, polarity, and same wavelength. The laser light is emitted with wavelengths of 600-900 nm, in an embodiment. The laser light is configured to be emitted at 850 nm, in an embodiment. The laser light is provided with a duration including the range of 1 μs to 30 μs, in an embodiment. The laser light is provided at one or more of multiple different pulses widths, including 10 μs, 20 μs, 40 μs, and 80 μs, in an embodiment.

At operation 1004, process 1000 includes capturing, with an image sensor, sequential images of an exit signal from the cranium at intervals, according to an embodiment. The exit signal may be captured using one or more detector optical fibers, a window, and one or more lenses. The detector optical fibers are multi-mode optical fibers having, for example, a core diameter that is greater than 10 μm. Examples of multi-mode optical fiber include (e.g., glass or plastic) optical fibers having a core diameter of 50 μm, 62.5 μm, 200 μm, 1 mm, or the like. In one embodiment, the one or more detector optical fibers are single-mode optical fibers, for example, having a core diameter of 9 μm or less.

At operation 1006, process 1000 includes determining coherence values of the sequential images, wherein each of the coherence values corresponds with one of the sequential images, according to an embodiment. Coherent light interference patterns are analyzed by determining a coherence value for each image. A coherence value may be a variance, a standard deviation, and/or speckle contrast of the image. Speckle contrast may be defined, for example, as dividing the standard deviation of the pixels of the image by the mean of the pixels of the image, in an embodiment.

At operation 1008, process 1000 includes combining the coherence values into a waveform, according to an embodiment. The waveform represents motion within a cranium. The waveform may be saved in a table, chart, database, or other data structure in coherence value and time interval (or time stamp) pairs. The waveform data may also include an indication of which light detector, light source, cranial location, wavelength, and distance between light source and light detector was used to generate the waveform. Lower coherence values represent more motion within the cranium, and higher coherence values represent less motion within the cranium. Motion may be attributed to blood cell motion, blood vessel motion, brain tissue motion, and/or cranial vibration.

At operation 1010, process 1000 includes inverting the waveform of coherence values into an inverted waveform that resembles an intracranial pressure (“ICP”) waveform, according to an embodiment. Inverting the waveform may include multiplying each coherence value by negative one (−1) and normalizing (e.g., dividing each value by the waveform mean) the inverted coherence values so that they range in values of 0 to 1, for example. The inverted waveform may then resemble an ICP waveform having three peaks per cycle, typically.

At operation 1012, process 1000 includes determining intracranial blood flow performance at least partially based on characteristics of the inverted waveform, according to an embodiment. Process 1000 may apply one or more of a number of rules (e.g., rules 591 of FIG. 5B) to the waveform data to determine if a cycle within the waveform represents reduced intracranial blood flow.

At operation 1014, process 1000 includes outputting a notification if the characteristics of the inverted waveform are indicative of potentially reduced intracranial blood flow, according to an embodiment. A notification may be output to a display, a speaker, a haptic motor, or to a computing device (e.g., via email, a file, etc.). The notification may include numerical data (e.g., blood flow rates, a blood flow index) and/or may include text-based messages (e.g., nominal blood flow, typical blood flow, unknown blood flow, and/or reduced blood flow), for example.

The term “processing logic” (e.g., processing logic 108, 508, 581, or 608) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Communication channels may include or be routed through one or more wired or wireless communication utilizing IEEE 802.11 protocols, Bluetooth, SPI (Serial Peripheral Interface), I²C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a smartwatch, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An imaging system comprising:
a laser configured to emit coherent light;
a source optical fiber coupled to the laser and configured to deliver the coherent light into a tissue sample within a cranium;
a detector optical element configured to receive an exit signal of the coherent light that exits the cranium;
an image sensor coupled to the detector optical element and configured to capture sequential images of the exit signal at intervals; and
processing logic configured to:
receive the sequential images from the image sensor;
determine coherence values of the sequential images, wherein each of the coherence values corresponds with one of the sequential images;
combine the coherence values into a waveform;
analyze characteristics of the waveform to identify intracranial blood flow performance; and
output a notification, if the characteristics of the waveform are indicative of reduced intracranial blood flow.

2. The imaging system of claim 1, wherein the coherence values include a standard deviation calculation of at least a portion of each of the sequential images of the exit signal.

3. The imaging system of claim 1, wherein the coherence values are inversely related to motion of the tissue sample and the cranium, wherein the tissue sample includes blood cells, blood vessels, and brain tissue.

4. The imaging system of claim 1, wherein the coherent light is pulsed laser light having a pulse duration ranging from 10 μs to 1000 μs.

5. The imaging system of claim 1, wherein the waveform is an array of coherence values associated with data values representing time stamps at which the sequential images are captured.

6. The imaging system of claim 1, wherein the coherent light is near-infrared laser light, and wherein the image sensor includes a filter to reduce light signals that are outside of a linewidth of the coherent light.

7. The imaging system of claim 1, wherein the waveform is a first waveform captured from a first location on the cranium, wherein the processing logic is configured to analyze characteristics of the first waveform with a comparison between the characteristics of the first waveform and characteristics of a second waveform, wherein the second waveform being concurrently captured from a second location on the cranium.

8. The imaging system of claim 1, wherein the processing logic is configured to analyze characteristics of the waveform based on whether a cycle of the waveform includes a first valley having a first depth and a second valley having a second depth, wherein the intracranial blood flow performance is defined as reduced if the second depth of the second valley has a value that is at least as low as the first depth of the first valley.

9. The imaging system of claim 1, wherein the processing logic is configured to analyze characteristics of the waveform based on at least one of: independent component analysis, artificial neural network models, or wavelet analysis.

10. The imaging system of claim 1, wherein the processing logic is configured to analyze characteristics of the waveform based on whether a cycle of the waveform includes more than one valley per cycle, wherein the intracranial blood flow performance is defined as reduced if the cycle of the waveform is limited to a single valley per cycle and a single peak.

11. The imaging system of claim 1, wherein the notification that is output is at least one of: a message on a display, a picture on a display, an audio alert, an audio message, a haptic pattern, or an electronic message.

12. An imaging method comprising:
emitting coherent light into a tissue sample in a cranium;
capturing sequential images of an exit signal from the cranium at intervals;
determining coherence values of the sequential images, wherein each of the coherence values corresponds with one of the sequential images;
combining the coherence values into a waveform;
determining intracranial blood flow performance at least partially based on characteristics of the waveform; and
outputting a notification, if the characteristics of the waveform are indicative of potentially reduced intracranial blood flow.

13. The imaging method of claim 12, wherein the waveform is an array of coherence values associated with data values representing the intervals at which the sequential images are captured.

14. The imaging method of claim 12, wherein the coherence values are inversely related to motion of the tissue sample.

15. The imaging method of claim 14, wherein the motion of the tissue sample includes one or more of: blood cell motion, blood vessel motion, and brain tissue motion, wherein the coherence values are inversely related to motion of the cranium combined with the motion of the tissue sample.

16. The imaging method of claim 12, wherein the waveform includes a cycle, wherein characteristics of the waveform include one or more of: a number of valleys in the cycle, relative values of adjacent valleys in the cycle, a number of peaks in the cycle, relative values of adjacent peaks in the cycle, and the coherence values of the waveform relative to second coherence values from a second waveform that is concurrently captured from the cranium.

17. An imaging method comprising:
emitting coherent light into a tissue sample in a cranium;
capturing, with an image sensor, sequential images of an exit signal from the cranium at intervals;
determining coherence values of the sequential images, wherein each of the coherence values corresponds with one of the sequential images;
combining the coherence values into a waveform;
inverting the waveform of coherence values into an inverted waveform that resembles an intracranial pressure (ICP) waveform;
determining intracranial blood flow performance at least partially based on characteristics of the inverted waveform; and
outputting a notification if the characteristics of the inverted waveform are indicative of potentially reduced intracranial blood flow.

18. The imaging method of claim 17, wherein the characteristics of the inverted waveform include a first peak, a second peak, and a third peak, wherein the first peak resembles a percussion wave of the ICP waveform, wherein the second peak resembles a tidal wave of the ICP waveform, wherein the third peak resembles a dicrotic wave of the ICP waveform.

19. The imaging method of claim 18, wherein intracranial blood flow performance is defined as potentially reduced, if an amplitude of the second peak is at least as great as an amplitude of the first peak.

20. The imaging method of claim 18, wherein intracranial blood flow performance is defined as potentially reduced, if the first peak is rounded such that the second peak and the third peak are indistinguishable from the first peak.

21. The imaging method of claim 18, wherein the inverted waveform is a first inverted waveform, wherein the sequential images are first sequential images captured at a first location on the cranium, wherein the imaging method further comprises:
capturing second sequential images of the exit signal from a second location on the cranium at the intervals;
forming a second inverted waveform from coherence values of the second sequential images; and
defining intracranial blood flow performance as potentially reduced, if a quantity of peaks of a cycle of the second inverted waveform are more or less than a quantity of peaks of a cycle of the first inverted waveform.
